(19) United States
(12) Patent Application Publication
(10) Pub. No.: US 2012/0045499 A1
(43) Pub. Date: Feb. 23, 2012

(54) PEARLESCENT PIGMENT SURFACE TREATMENT FOR CONFECTIONERY

(75) Inventors: Juan Pablo Campomanes Marin, Puebla (MX); Hector Octavio Vilches Landin, Mexico City (MX)

(73) Assignee: CADBURY ADAMS MEXICO, S. DE R.L. DE C.V., Delegacion Cuajimalpa , Mexico D.F. (MX)

(21) Appl. No.: 13/142,918

(22) PCT Filed: Dec. 30, 2009
(86) PCT No.: PCT/IB09/08077

§ 371 Date: Nov. 9, 2011

Related U.S. Application Data
(60) Provisional application No. 61/141,678, filed on Dec. 31, 2008.

(51) Int. Cl. A23G 3/56; A23L 1/30; A23L 1/275; A23P 1/08; A61P 3/00; A23G 3/24; A61K 9/00; A61P 25/04; A61P 1/04; A61P 11/04; A23G 3/54; A23G 3/20

(52) U.S. Cl. 424/440; 426/103; 426/250; 118/23

(57) ABSTRACT

A confection with unique visual impression is created by partially treating the surface area of the confection with a coating composition including a pearlescent pigment such that the initial taste perception of the confection is not adversely affected by the coating composition.

### CROSS REFERENCE TO RELATED APPLICATIONS

This application is a U.S. national stage of application No. PCT/IB2009/008077, filed on Dec. 30, 2009, which claims priority to U.S. Application Ser. No. 61/141,678, filed on Dec. 31, 2008. The disclosures of these related applications are incorporated herein by reference.

### FIELD

The present invention relates to confectionery compositions with pigmented coating compositions that include pearlescent pigments and which do not adversely impact the initial taste perception of the confectionery compositions. The present invention also includes methods of making such confectionery compositions.

### BACKGROUND

Confectionery products with visual effects such as swirled color patterns are known. Surface treatments that provide visual effects such as panning with food colors are also known. Pearlescent pigments are a relatively recent development in the area of food colors. However, the use of pearlescent pigments as components of confectionery surface treatments has resulted in taste problems including off-tastes and delayed initial taste perception of the confectionery product. Therefore, it would be desirable to have confectionery products with the visual appeal of pearlescent pigments but without the taste problems.

### SUMMARY

The present invention relates to a confectionery composition comprising a hard candy substrate having a total surface area, and a pigmented coating composition including a pearlescent pigment and a fluid carrier, wherein the pigmented coating composition covers less than substantially 75%, preferably less than substantially 50%, and more preferably less than substantially 10% of said total surface area. For some embodiments, the amount of total surface area covered can be less than substantially 75%, 70%, 65%, 60%, 55%, 50%, 45%, 40%, 35%, 30%, 25%, 20%, 15%, 10%, 5%, or 2%. In some embodiments the amount of total surface area can be between substantially 2%-75%, 2%-70, 2%-65%, 5%-75%, 10%-75%, 2%-50%, 5%-50%, 10%-50%, 2%-10%, or 5%-10%.

The present invention also relates to a confectionery composition comprising a hard candy substrate having a total surface area, and a pigmented coating composition including a powdered pearlescent pigment, wherein the pigmented coating composition covers less than substantially 75%, preferably less than substantially 50%, and more preferably less than substantially 10% of said total surface area. For some embodiments, the amount of total surface area covered can be less than substantially 75%, 70%, 65%, 60%, 55%, 50%, 45%, 40%, 35%, 30%, 25%, 20%, 15%, 10%, 5%, or 2%. In some embodiments the amount of total surface area can be between substantially 2%-75%, 2%-70%, 2%-65%, 5%-75%, 10%-75%, 2%-50%, 5%-50%, 10%-50%, 2%-10%, or 5%-10%.

Hard candy substrates of the present invention can include boiled candies, toffees, and the like. In some embodiments, the hard candy substrate can be a lollipop. In some embodiments, the hard candy substrate can include a multi-region format such as a center-filled format.

Hard candy substrates of the present invention can include those with porous surfaces. For hard candy substrates with porous surfaces, the pigmented coating composition can include a powdered pearlescent pigment without a fluid carrier. In some embodiments, the pigmented coating composition for use with a hard candy substrate with a porous surface can include a powder carrier. In some embodiments, the powder carrier can include ingredients such as, but not limited to, mannitol, talc, isomalt, maltitol, polyglycitols, lactitol, erythritol, sucrose, and combinations thereof. In some embodiments, powdered pearlescent pigments with average particle sizes of from substantially 10 microns to substantially 250 microns can be used. In some embodiments, the amount of powdered pearlescent pigment included on a porous hard candy substrate can range from substantially 0.01% w/w to substantially 0.5% w/w by weight of the hard candy substrate.

Hard candy substrates of the present invention can be transparent or translucent. For transparent or translucent hard candy substrates, a desirable visual effect can be created by including pearlescent pigments with an average particle size of from substantially 10 microns to substantially 250 microns in the hard candy substrate and then including pigmented coating compositions with either powdered pearlescent pigments and a powder carrier or pigmented coating compositions with pearlescent pigments and a fluid carrier on at least one of the surfaces of the hard candy substrate.

In some embodiments, the confectionery composition includes more than one surface region. In such embodiments, surface area coverage of less than substantially 75% can be achieved when the pigmented coating composition covers 100% of one of the more than one surface region. For example, where the confectionery composition includes two surface regions and one of the coating compositions covers 100% of one of the two surface regions, the coated surface region can represent less than substantially 75%, 70%, 65%, 60%, 55%, 50%, 45%, 40%, 35%, 30%, 25%, 20%, 15%, 10%, 5%, or 2% of the total surface area of the confectionery composition.

In addition to the pearlescent pigment, the pigmented coating composition can include a range of other ingredients. In some embodiments, the pigmented coating composition can include least one food color selected from the group consisting of artificial colors, natural colors, and combinations thereof.

In other embodiments, the pigmented coating composition can include at least one tastant selected from the group consisting of flavors, sweeteners, acids, salts, potentiators, and combinations thereof.

In still other embodiments, the pigmented coating composition can include at least one active selected from the group consisting of medicaments, nutraceuticals, pharmaceuticals, micronutrients, and combinations thereof.

In addition to the pearlescent pigment and optional ingredients, the pigmented coating composition can include a fluid carrier. Due to the moisture sensitivity of hard candy substrates, the fluid carrier of the present invention will desirably a low moisture content. In some embodiments, the fluid carrier is selected from the group consisting of oil, fat, wax, shellac, and combinations thereof.

According to the invention, an advantageous visual effect can be created when the pigmented coating composition forms a pattern. A range of patterns and combinations of patterns are contemplated by the invention. In some embodiments, the pigmented coating composition forms a striped pattern, while in other embodiments, the pigmented coating composition forms a swirled pattern, while in still other embodiments, the pigmented coating composition forms a random pattern.

In some embodiments, a desirable visual effect can be created when the pigmented coating composition forms a striped pattern combined with a swirled pattern, or when the pigmented coating composition forms a striped pattern with a random pattern, or when the pigmented coating composition forms a swirled pattern with a random pattern, or when the pigmented coating composition forms a striped pattern combined with a swirled pattern and a random pattern, or any combination thereof. Additionally, in some embodiments, a striped or swirled pattern can be created by random patterned objects.

In some embodiments, the particle size of the pearlescent pigment can determine the visual effect. For example, in some embodiments, pearlescent pigments with average particle size of less than substantially 15 microns can create a silky-looking appearance. In other embodiments, pearlescent pigments with average particle size from substantially 5 microns to substantially 25 microns can create a satin-looking appearance. In still other embodiments, pearlescent pigments with average particle size from substantially 10 microns to substantially 60 microns can create a metallic-looking appearance. In yet other embodiments, pearlescent pigments with average particle size from substantially 10 microns to substantially 250 microns can create a glitter-looking appearance.

In some embodiments, there is provided a confectionery composition comprising a hard candy substrate and a pigmented coating composition having a total surface area and an initial flavor intensity prior to coating and a final flavor intensity subsequent to coating, wherein the pigmented coating composition covers less than substantially 75% of said total surface area and the initial flavor intensity is substantially the same as the final flavor intensity.

In some embodiments, the confectionery compositions including a hard candy substrate and a pigmented coating composition have an initial flavor intensity and a final flavor intensity. The flavor intensities are measured on a linear scale with integers representing increasing levels of flavor intensities. In some embodiments, the initial flavor intensity is from substantially 6 to substantially 8 on a linear scale of 1 to 10. In other embodiments, the final flavor intensity is from substantially 6 to substantially 8 on a linear scale of 1 to 10. In still other embodiments, the difference between the initial flavor intensity and the final flavor intensity is less than substantially 1 on a scale of 1 to 10 and is preferably less than substantially 0.5 on a scale of 1 to 10.

The present invention further relates to a method of applying a pigment to a confection comprising the steps of providing a hard candy substrate having a total surface area, providing a pigmented coating composition including a pearlescent pigment and a fluid carrier, and applying the pigmented coating composition to the hard candy substrate such that the pigmented coating composition covers less than substantially 75% of the total surface area, and wherein the applying step involves a process other than panning. In some embodiments, the amount of total surface area covered by the method can be less than substantially 75%, 70%, 65%, 60%, 55%, 50%, 45%, 40%, 35%, 30%, 25%, 20%, 15%, 10%, 5%, or 2%.

The present invention further relates to an apparatus for applying pigment to a confection comprising a confectionery substrate conveyor and a pigment applicator, wherein the pigment applicator is selected from the group consisting of sprayers, brushes, rollers, dippers, stencils, and combinations thereof and wherein the pigment applicator covers a confectionery substrate having a total surface area with less than substantially 75% coverage of said total surface area. In some embodiments, the amount of total surface area covered by the apparatus can be less than substantially 75%, 70%, 65%, 60%, 55%, 50%, 45%, 40%, 35%, 30%, 25%, 20%, 15%, 10%, 5%, or 2%.

In some embodiments, the pigmented coating composition of the method and/or apparatus covers less than substantially 50% of said total surface area.

In some embodiments, the method and/or apparatus includes applying the pigmented coating composition by spraying and the spraying includes intermittent spraying.

In some embodiments, the method and/or apparatus includes applying using brushing and the brushing includes providing one or more brushes including a set of bristles wherein the bristles are selected from the group consisting of bristles arranged to include spaces, bristles arranged to include spaces that are at least 1 mm apart, and combinations thereof.

In some embodiments, the method and/or apparatus includes applying using stenciling and the stenciling includes providing one or more stencils wherein the one or more stencils block no more than 25% of the total surface area during the applying.

In some embodiments, the method applying step is selected from the group consisting of spraying, brushing, rolling, dipping, stenciling, and combinations thereof.

Is some embodiments, the method includes a forming step prior to the applying step. In other embodiments, the method includes a forming step subsequent to the applying step.

### DETAILED DESCRIPTION

The initial taste and flavor perception of confectionery products such as hard candies that rely on sucking or licking action for consumption can be adversely affected by surface treatments that cover the surface area of the confection and thus interfere with or delay that initial taste impression. Unlike confections where chewing action provides the initial taste perception, sucking/licking candies require an outer surface area that provides a positive taste perception. Sucking/licking candies are also moisture sensitive and thus pigments designed for application to the surface area of sucking candies are generally provided in non-aqueous carrier systems. When these pigments in their carrier systems are applied to the outer surface area of sucking/licking candies, they can interfere with the candies' taste impression by imparting off-tastes such as oily notes and/or they can delay or decrease the intensity of the initial taste perception of the candy. Attempting to minimize the adverse taste affect by applying too little pigment to the candies' surface can result in a muted or unattractive visual effect. The inventors here have surprisingly found that confectionery compositions with pigmented coating compositions including pearlescent pigments and fluid carriers covering only part of the candies' surface area effectively balance desirable visual effects with desirable taste perception.

The inventors have also surprisingly found that powdered pearlescent pigments can be used on hard candy substrates with porous surfaces without a fluid carrier. The porosity of the hard candy substrate surface enables retention of an amount of powdered pearlescent pigment that effectively balances desirable visual effects with desirable taste perception. Porous hard candy substrate surfaces can be formed by pulling or aerating the hard candy substrate. Porosity of the hard candy substrate can be increased by increased pulling and/or by the incorporation of more air. Increased pulling and/or incorporation of more air can create larger voids in the surface of the hard candy substrate which can, in turn, accommodate larger particle sizes of powdered pearlescent pigment. In some embodiments, powdered pearlescent pigment with average particle sizes of from substantially 10 microns to substantially 250 microns can be used. In some embodiments, the amount of powdered pearlescent pigment included on a porous hard candy substrate can range from substantially 0.01% w/w to substantially 0.5% w/w by weight of the hard candy substrate.

As used herein, the term “hard candy substrate” refers to confectionery compositions with moisture contents of less than 5% where the initial taste perception of the confectionery compositions results from sucking or licking action. Hard candy substrates are generally prepared by reducing the moisture of saccharide syrups (including sugar and sugarless saccharide syrups) as through heating. Hard candy substrates can include, but are not limited to, lollipops, boiled candies, toffees, hard caramels, and the like.

As used herein, the term “lollipop” refers to confectionery compositions with moisture contents of less than 5% where the initial taste perception of the confectionery compositions results from sucking or licking action and the confectionery composition includes a handle. As used herein, the term “handle” refers to any structure capable of supporting the lollipop during eating. Handles can include, but are not limited to, sticks, strings, ropes, rings, and the like which can be constructed from edible materials, paper, plastic, or composite materials.

As used herein, the term “pigmented coating composition” refers to a blend of ingredients that impart at least visual effects when applied to the surface of a confectionery composition. Such ingredients can include food colors, pearlescent pigments, optical effect materials such as those that create holographic effects or bioluminescent effects, and the like and combinations thereof. The pigmented coating composition can also include ingredients that facilitate application of the pigmented coating composition to the surface of the confectionery. Such ingredients can include fluid carriers such as oils, fats, waxes, shellacs, and the like and combinations thereof. The pigmented coating composition can also include ingredients that impart taste or functionality such as sweeteners, acids, salts, potentiators, actives, and the like and combinations thereof.

As used herein, the term “pearlescent pigment” refers to materials that exhibit a spectrum of colors resulting from light refraction instead of pigmentation where color results from light absorption. These “pearlescent pigments” exhibit colors depending on the angles of illumination. A pearlescent pigment material generally creates a glossy finish appearance, rather than a matte finish. Pearlescent pigments can be based on inorganic layers or coatings formed on substrates such as glass or potassium aluminum silicate (also known as mica) through calcination. In some embodiments, the pearlescent pigment can be formed by depositing titanium dioxide and/or iron oxide onto mica platelets with or without other colorants. As used herein, the term “powdered pearlescent pigment” refers to a pearlescent pigment in particulate form without a fluid carrier. In some embodiments, a pigmented coating composition can include a powdered pearlescent pigment and a powder carrier. In some embodiments, a powder carrier can include ingredients such as, but not limited to, mannitol, talc, isomalt, maltitol, polyglycitols, lactitol, erythritol, sucrose, and combinations thereof.

Turning to the hard candy substrate of the invention, these confectionery compositions are generally characterized for all aspects of the invention by their moisture content and initial taste perception. Hard candy substrates generally have moisture contents of less than 5%. In some embodiments, the hard candy substrate has a moisture content of substantially less than 3%, preferably substantially less than 2%, and more preferably substantially between 0.2% and 2%. In some embodiments, the hard candy substrate has a moisture content of substantially between 0.2% and 5% or substantially between 0.2% and 3%. As to initial taste perception, hard candy substrates generally involve sucking or licking action such that an initial taste perception is created based on the sucking or licking of the hard candy substrate's surface. Non-limiting examples of hard candy substrates can include lollipops, boiled candy including drops, balls, lozenges, and the like, toffees, hard caramel, jawbreakers, and the like and combinations thereof.

In some embodiments, the hard candy substrate can include a multi-region format. In some embodiments, multi-region formats can include layered formats with multiple exterior surfaces such as, but not limited to, “sandwich” formats. In other embodiments, multi-region formats can include layered formats without multiple such as, but not limited to, center-filled formats where the interior region(s) can be liquid, powder, and the like.

In some embodiments, the hard candy substrate can include more than one surface region. For example, the hard candy substrate can have a top surface region and a bottom surface region or an upper surface region and a lower surface region and the like. In such embodiments, surface area coverage of less than substantially 75% can be achieved when the pigmented coating composition covers 100% of one of the more than one surface regions. For example, where the confectionery composition includes two surface regions and one of the coating compositions covers 100% of one of the two surface regions, the coated surface region can represent less than substantially 75%, 70%, 65%, 60%, 55%, 50%, 45%, 40%, 35%, 30%, 25%, 20%, 15%, 10%, 5%, or 2% of the total surface area of the confectionery composition.

Hard candy substrates can include conventional confectionery ingredients such as, but not limited to sweeteners, flavors, sensates, colors, acids, salts, potentiators, actives, and the like.

Suitable sweeteners can include, but are not limited to, sugars, sugarless bulk sweeteners, or the like, high intensity sweeteners, or mixtures thereof. Suitable sugar sweeteners generally include monosaccharides, disaccharides and polysaccharides such as but not limited to, sucrose (sugar), dextrose, maltose, dextrin, xylose, ribose, glucose, mannose, galactose, fructose (levulose), invert sugar, sucromalt, corn syrups, maltodextrins, oligosaccharide syrups, fructooligosaccharide syrups, partially hydrolyzed starch, corn syrup solids, resistant starches, and mixtures thereof.

Suitable sugarless bulk sweeteners include sugar alcohols (or polyols) such as, but not limited to, sorbitol, xylitol, mannitol, galactitol, maltitol, hydrogenated isomaltulose (ISOMALT), lactitol, erythritol, hydrogenated starch hydrolysates, and mixtures thereof.

In some embodiments, high-intensity sweeteners also may be included as sweetening agents in the compositions. Without being limited to particular sweeteners, representative categories include:

(a) water-soluble sweetening agents such as dihydrochalcones, monellin, steviosides and stevia derived compounds such as but not limited to rebaudioside A, iso-mogroside V and the like, lo han quo and lo han quo derived compounds, glycyrrhizin, dihydroflavenol,

(b) water-soluble artificial sweeteners such as soluble saccharin salts, Acesulfame-K, the free acid form of saccharin, and mixtures thereof;

(c) dipeptide based sweeteners, Aspartame, Neotame, and materials described in U.S. Pat. No. 3,492,131, Alitame, and mixtures thereof;

(d) aspartame-acesulfame salts, known, for example under the product designation of TwinSweet™:

(e) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose or Splenda™; and mixtures thereof;

(f) protein based sweeteners such as thaumaoccous danielli (Thaumatin I and II) and talin;

(g) the sweetener monatin (2-hydroxy-2-(indol-3-ylmethyl)-4-aminoglutaric acid) and its derivatives or isomers; and

(h) the sweetener Lo han guo (sometimes also referred to as “Lo han kuo”).

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In addition to sweeteners, the hard candy substrates can include flavors. Suitable flavors can include, but are not limited to, synthetic flavor oils and flavoring aromatics and/or oils, oleoresins and extracts derived from plants, leaves, flowers, fruits, and so forth, and combinations thereof. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

In some embodiments, flavoring agents are used at levels that provide a perceptible sensory experience i.e. at or above their threshold levels. In other embodiments, flavoring agents are used at levels below their threshold levels such that they do not provide an independent perceptible sensory experience. At subthreshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

In addition to these potential flavoring agents, the hard candy substrates can also include sensate ingredients. Sensate compounds can include cooling agents, warming agents, tingling agents, effervescent agents, and combinations thereof A variety of well known cooling agents may be employed. For example, among the useful cooling agents are included xylitol, erythritol, dextrose, sorbitol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), ethyl ester of N-[[5-methyl-2-(1-methylethyl)cyclohexyl]carbonyl]glycine (WS5).

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamylether, vanillyl alcohol n-hexylether, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, jambu oleoresin, or spilanthol to some examples. In some embodiments, alkylamides extracted from materials such as jambu or sanshool can be included.

Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates, and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid, aspartic acid, benzoic acid, caffeotannic acid, iso-citric acid, citramalic acid, galacturonic acid, glucuronic acid, glyceric acid, glycolic acid, ketoglutaric acid, a-ketoglutaric acid, lactoisocitric acid, oxalacetic acid, pyruvic acid, quinic acid, shikimic acid, succinic acid, tannic acid, hydroxyacetic acid, suberic acid, sebacic acid, azelaic acid, pimelic acid, capric cid, and combinations thereof. Examples of “tingling” type sensates can be found in U.S. Pat. No. 6,780,443, the entire contents of which are incorporated herein by reference for all purposes.

In some embodiments, sensate components are used at levels that provide a perceptible sensory experience i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

The hard candy substrates can also include food colors and pearlescent pigments as described below. Various visual effects can be created by using these food colors and/or pearlescent pigments in either or both of the hard candy substrate and pigmented coating composition. For example, the hard candy substrate can include a food color that matches or has a similar hue to the food color included in the pigmented coating composition. In other examples, the food colors can be contrasting. In some embodiments, the hard candy substrate can be transparent or translucent. For transparent or translucent hard candy substrates, a desirable visual effect can be created by including pearlescent pigments with an average particle size of from substantially 10 microns to substantially 250 microns in the hard candy substrate and then including pigmented coating compositions with either powdered pearlescent pigments and a powder carrier or pigmented coating compositions with pearlescent pigments and a fluid carrier on at least one of the surfaces of the hard candy substrate.

In addition to sweeteners, flavors, colors, and sensates, the hard candy substrates can include acids. Suitable acids can include, but are not limited to, acetic acid, adipic acid, ascorbic acid, butyric acid, citric acid, formic acid, fumaric acid, glyconic acid, lactic acid, phosphoric acid, malic acid, oxalic acid, succinic acid, tartaric acid, aspartic acid, benzoic acid, caffeotannic acid, iso-citric acid, citramalic acid, galacturonic acid, glucuronic acid, glyceric acid, glycolic acid, ketoglutaric acid, a-ketoglutaric acid, lactoisocitric acid, oxalacetic acid, pyruvic acid, quinic acid, shikimic acid, succinic acid, tannic acid, hydroxyacetic acid, suberic acid, sebacic acid, azelaic acid, pimelic acid, capric acid, and combinations thereof.

Further, the hard candy substrates can include salts. Suitable salts can include, but are not limited to, sodium chloride, potassium chloride, sea salt, and combinations thereof.

Still further, the hard candy substrates can include potentiators. Suitable potentiators can include, but are not limited to, materials that may intensify, supplement, modify or enhance the taste and/or aroma perception of an original material without introducing a characteristic taste and/or aroma perception of their own. In some embodiments, potentiators designed to intensify, supplement, modify, or enhance the perception of flavor, sweetness, tartness; umami, kokumi, saltiness, and combinations thereof can be included.

In some embodiments, examples of suitable potentiators, also known as taste potentiators include, but are not limited to, neohesperidin dihydrochalcone, optically active S-alkyl 2-methyl butane thioate compounds such as those disclosed in PCT Application Number WO 2007/032262 to Ogura et al, which is incorporated in its entirety herein by reference, chlorogenic acid, alapyridaine, cynarin, miraculin, glupyridaine, pyridinium-betain compounds, glutamates, such as monosodium glutamate and monopotassium glutamate, neotame, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(1-carboxyethyl)-6-(hydroxymethyl)pyridinium-3-ol inner salt, compositions comprising 5′-nucleotides such as those disclosed in US 2006/0078972 to Noordam et al, which is incorporated in its entirety herein by reference, sugar beet extract (alcoholic extract), sugarcane leaf essence (alcoholic extract), curculin, strogin, mabinlin, gymnemic acid, hydroxybenzoic acids, 3-hydrobenzoic acid, 2,4-dihydrobenzoic acid, citrus aurantium, vanilla oleoresin, sugarcane leaf essence, maltol, ethyl maltol, vanillin, licorice glycyrrhizinates, compounds that respond to the TRPMS ion channel that mediates taste receptors for sweet, bitter, and savory tastes as disclosed in U.S. Patent Application Number 2005/0019830 to Penner et al., which is incorporated in its entirety herein by reference, pyridinium betain compounds as disclosed in U.S. Pat. No. 7,175,872 to Hofmann et al., which is incorporated in its entirety herein by reference, compounds that respond to G-protein coupled receptors (T2Rs and T1Rs) and taste potentiator compositions that impart kokumi, as disclosed in U.S. Pat. No. 5,679,397 to Kuroda et al., which is incorporated in its entirety herein by reference. “Kokumi” refers to materials that impart “mouthfulness” and “good body”.

Sweetener potentiators, which are a type of taste potentiator, enhance the taste of sweetness. In some embodiments, exemplary sweetener potentiators include, but are not limited to, monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, hydroxybenzoic acids, tagatose, trehalose, gurmarin, extracts and derivatives of extracts from Gymnema sylvestre, tryptophan, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs), compounds that respond to the human T1R2 taste receptor, and combinations thereof.

Additional examples of potentiators for the enhancement of salt taste include acidic peptides, such as those disclosed in U.S. Pat. No. 6,974,597, herein incorporated by reference. Acidic peptides include peptides having a larger number of acidic amino acids, such as aspartic acid and glutamic acid, than basic amino acids, such as lysine, arginine, and histidine. The acidic peptides are obtained by peptide synthesis or by subjecting proteins to hydrolysis using endopeptidase, and if necessary, to deamidation. Suitable proteins for use in the production of the acidic peptides or the peptides obtained by subjecting a protein to hydrolysis and deamidation include plant proteins, (e.g. wheat gluten, corn protein (e.g., zein and gluten meal), soybean protein isolate), animal proteins (e.g., milk proteins such as milk casein and milk whey protein, muscle proteins such as meat protein and fish meat protein, egg white protein and collagen), and microbial proteins (e.g., microbial cell protein and polypeptides produced by microorganisms).

The sensation of warming or cooling effects may also be prolonged with the use of a hydrophobic sweetener as described in U.S. Patent Application Publication 2003/0072842 A1 which is incorporated in its entirety herein by reference.

Perillartine may also be added as described in U.S. Pat. No. 6,159,509 also incorporated in its entirety herein by reference.

Additionally, the hard candy substrates can include actives. Suitable actives can include medicaments, nutraceuticals, pharmaceuticals, micronutrients, and combinations thereof.

Suitable medicaments, can include but are not limited to, analgesics, antacids, throat soothing agents, appetite suppressors, and combinations thereof.

Suitable nutraceuticals can include, but are not limited to, herbals which are generally aromatic plants or plant parts and or extracts thereof that can be used medicinally or for flavoring. Suitable herbals can be used singly or in various mixtures.

Suitable micronutrients can include virtually any vitamin or mineral.

Turning to the pigmented coating composition of the invention, these compositions are designed to create surface treatment effects including pearlescence, color, gloss, and the like and combinations thereof. As described above, pearlescence refers to the optical effect created by light refraction. Gloss refers to the shine quality of a surface treatment. Pigmented coating compositions can be created to include all or only some of these effects. In some embodiments it is desirable to provide only the pearlescent effect while in other embodiments it is desirable to provide the pearlescent effect along with either one or more colors or gloss while in still other embodiments it is desirable to provide the pearlescent effect along with color and gloss. In some embodiments, the particle size of the pearlescent pigment can determine the visual effect. For example, in some embodiments, pearlescent pigments with average particle size of less than substantially 15 microns can create a silky-looking appearance. In other embodiments, pearlescent pigments with average particle size from substantially 5 microns to substantially 25 microns can create a satin-looking appearance. In still other embodiments, pearlescent pigments with average particle size from substantially 10 microns to substantially 60 microns can create a metallic-looking appearance. In yet other embodiments, pearlescent pigments with average particle size from substantially 10 microns to substantially 250 microns can create a glitter-looking appearance.

Pigmented coating compositions can be formulated to provide a number of characteristics such as water solubility, viscosity, tackiness, stability, and the like. As described in PCT publication No. WO 2008/076902 which is incorporated herein by reference for all purposes, pigmented coating compositions can include optional such as viscosity modifiers, detacking agents, stabilizers, and thickeners. These optional ingredients help prevent separation of the pigmented coating compositions. In particular, these optional ingredients can help prevent settling out of the insoluble pigments. Additionally, the pigmented coating compositions aid processability including improving application of the pigmented coating composition to the surface of a food product.

Pigmented coating compositions are commercially available from a number of suppliers including Sensient Colors Inc., Merck KGaA, BASF, HebeiOxen, and Watson Foods Inc.

As described above, pearlescent pigments are those that exhibit a spectrum of colors resulting from light refraction. Pearlescent pigments can include materials such as those describe in U.S. Patent Publication No. 2007/0020208 which is incorporated herein by reference for all purposes, where the pearlescent pigment is modified through chemical bonding to create a pearlescent pigment with the desired optical effect. Other pearlescent pigments are described in U.S. Patent Publication No. 2008/0014321 which is incorporated herein by reference for all purposes and include a range of platy substrates coated with titanium dioxide and/or iron oxides.

Pearlescent pigments are commercially available under a number of trade names including SensiPearl™, Candurin™, and Watson Food's Edible Glitter™. Suitable pearlescent pigments such as those available under the Candurin™ trade name can be supplied to provide a wide range of color effects including, but not limited to, silver fine, silver sheen, silver lustre, silver sparkle, gold shimmer, red shimmer, blue shimmer, green shimmer, gold sheen, light gold, gold lustre, brown amber, orange amber, red amber, red lustre, and red sparkle.

Pearlescent pigments can include natural and/or artificial food colors. Exemplary food colors considered to be natural can include, but are not limited to, annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), indigo carmine, carmoisine/azorubine (E122), sodium copper chlorophyllin (E141), chlorophyll (E140), chlorophyllin, copper chlorophyllin, copper chlorophyll, curcumin/copper chlorophyllin, toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot, carrot oil, corn endosperm oil, paprika, paprika oleoresin, paprika extract, phaffia yeast, riboflavin (E101), saffron, turmeric (E100), turmeric oleoresin, tumeric root, amaranth (E123), capsanthin/capsorbin (E160c), lycopene (E160d), black carrot, black carrot extract, elderberry, aronia, allura red, curcumin, spinach, stinging nettle, burnt sugar, carbo medicinalis, and combinations thereof.

Food colors considered to be artificial can include but are not limited to, certified colors such as FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), brilliant blue FCF, titanium dioxide (E171), aluminium (E173), allura red, silver (E174), gold (E175), pigment rubine/lithol rubine BK (E180), calcium carbonate (E170), carbon black (E153), black PN/brilliant black BN (E151), brown HT, green S, green S/acid brilliant green BS (E142), and combinations thereof. In some embodiments, certified colors can include FD&C aluminum lakes. These consist of the aluminum salts of FD&C dyes extended on an insoluble substrate of alumina hydrate. Additionally, in some embodiments, certified colors can be included as calcium salts.

Turning to the fluid carrier of the invention, as described in PCT publication No. WO 2008/076902, fluid carriers can include, but are not limited to, 1,3-butylene glycol, 1,1,2-trichlorotrifluoroethane, 2-nitropropane, acetone, acetylated monoglycerides, amyl acetate, benzyl alcohol, butan-1-ol, butan-2-ol, castor oil, coconut oil, cottonseed oil, dichloromethane, diethyl ether, diethyl tartrate, diethylene glycol monoethyl ether, food grade oils, glucose syrup, ethyl acetate, ethyl alcohol, ethyl methyl ketone, ethylene dichloride, furfural, glycerin, glycerol, glycerol diacetate, heptane, hexabe, isobutanol, isopropyl acetate, isopropyl alcohol, isopropyl myristate, maltitol, mannitol, medium chain triglycerides, methanol, methyl alcohol, methylene chloride, palm kernel oil, Poloxamer 331, Poloxamer 407, polyols, propan-1-ol, propan-2-ol, propylene glycol, SDA alcohol, sorbitols, soybean oil, sucrose syrup, syrups, toluene, triacetin, trichloroethylene, triethyl citrate, glucose syrup, corn syrup solids, xylitol, and combinations thereof.

Because the fluid carrier forms part of the pigmented coating composition which is applied to the surface of a hard candy substrate, it is advantageous to use fluid carriers that contribute little to no moisture to the pigmented coating composition.

In addition to pearlescent pigment(s) and fluid carrier(s), the pigmented coating composition of the invention can also include optional ingredients such as food color(s), tastant(s), active(s), and combinations thereof. Suitable food colors include those described above.

Suitable tastant(s) can include, but are not limited to, flavors, sweeteners, acids, salts, potentiators, and combinations thereof. Suitable flavors, sweeteners, acids, salts, potentiators can include those described above as components of the hard candy substrates.

In some embodiments, the sweeteners, flavors, sensates, acids, salts, potentiators, and/or actives can be in either or both of the hard candy substrates and pigmented coating composition to create a range of taste and functional benefits. For example, the hard candy substrate can include a cooling sensate while the pigmented coating composition can include a warming sensate. In another example, the hard candy substrate can include an acid that provides a tart taste perception while the pigmented coating composition includes a micronutrient such as vitamin C. In yet another example, the hard candy substrate can include a high-intensity sweetener to provide a sweet taste perception while the pigmented coating composition includes one or more acids to provide a tart taste perception.

In some embodiments of the invention, the pigmented coating composition is applied to the surface of a hard candy substrate in such a way as to create a pattern. Such patterns can be selected from the group consisting of a striped pattern, a swirled pattern or a random pattern, or any combination thereof. Suitable striped patterns can include those created by parallel, linear, or curvilinear lines. Suitable swirled patterns can include those created by non-parallel, curvilinear lines. Suitable random patterns can include any variety and/or shape of shaped objects such as, but not limited to, dots, stars, circles (open or closed), splotches, etc. Random patterns are generally considered to be those that do not provide a repeating series of objects. Combinations of patterns are also contemplated by the invention such as stripes with swirls, stripes with random objects, and swirls with random objects. Stripes and swirls created by random patterned objects are also contemplated.

In some embodiments, the initial taste perception of the confectionery composition with a pigmented coating composition is the same as a confectionery composition without a pigmented coating composition. This lack of diminished initial taste perception is achieved when the pigmented coating composition covers less than substantially 75% of the total surface area of the hard candy substrate.

Taste perception is generally considered to include both taste and flavor attributes. There are five primary categories of taste attributes that are sensed by humans: sour, salty, sweet, and bitter and umami (savory or the taste of glutamate). The taste of a substance is sensed by taste receptor cells located in taste buds primarily on the surface of the tongue and palate in the oral cavity. Each of the primary taste attributes is sensed by a specific mechanism. It is believed that sour and salty taste attributes are detected by the passage of ions, hydrogen and sodium respectively, through the ion channels in taste bud cells. This triggers a nerve impulse that is sensed in the brain as sour or salty. In contrast, it is believed that sweet, bitter and umami taste attributes are perceived by physical binding to receptors. In general, sweet, bitter and umami sensing taste cells have G-protein coupled receptors (GPCRs) on their surface. These receptors are activated when they bind to tastants, which initiates a series of signaling events that trigger a nerve impulse that is sensed in the brain as sweet, bitter, or savory.

Flavor is generally considered to be the combined perception a product creates in the mouth. Flavor attributes are measured by measuring the aromatics that characterize a flavor. Aromatics are the volatile components of the product that are perceived by the olfactory system. Measurement of specific flavor attributes characterized by specific aromatics can be used to compare products and to determine how well a product performs relative to a desired objective.

Taste and flavor attributes can be reliably and reproducibly measured using sensory analysis methods known as descriptive analysis techniques. The Spectrum™ method of descriptive analysis is described in MORTEN MEILGAARD, D.SC. ET AL., SENSORY EVALUATION TECHNIQUES (3d ed. 1999). The Spectrum™ method is a custom design approach meaning that the highly trained panelists who generate the data also develop the terminology to measure the attributes of interest. Further, the method uses intensity scales created to capture the intensity differences being investigated. These intensity scales are anchored to a set of well-chosen references. Using these references helps make the data universally understandable and usable over time. This ability to reproduce the results at another time and with another panel makes the data potentially more valuable than analytical techniques which offer similar reproducibility but lack the ability to fully capture the integrated sensory experiences as perceived by humans.

Sensory methods such as descriptive analysis can be conducted on a screening basis or on a full panel basis. When testing is done on a screening basis, there may be fewer panelists and those panelists may use an abbreviated scale such as a 5 point scale for rating the products. When testing is done on a full panel basis, more panelists may be involved and a complete scale such as a 10 or 15 point scale may be used. When choosing whether to use a screening or full panel basis for the testing, sensory scientists consider a range of variables such as cost, time, number of samples, etc. A screening basis may be used when time for generating the data is limited and/or when data from an abbreviated scale will adequately distinguish the samples.

The taste and flavor intensities can be measured on a linear scale with integers representing increasing levels of flavor intensities. In some embodiments, the initial flavor intensity is from substantially 6 to substantially 8 on a linear scale of 1 to 10. In other embodiments, the final flavor intensity is from substantially 6 to substantially 8 on a linear scale of 1 to 10. In still other embodiments, the difference between the initial flavor intensity and the final flavor intensity is less than substantially 1 on a scale of 1 to 10 and is preferably less than substantially 0.5 on a scale of 1 to 10.

In some embodiments, the total surface area of the confectionery composition includes more than one surface region. The more than one surface regions can be portions of a single confectionery mass or they can be separate confectionery masses. For example, an oval-shaped hard candy substrate such as a typical throat lozenge can have two relatively flat sides such as a top side and a bottom side. Each side can have a surface region contributing to the total surface area. In embodiments where one of the surface regions represented by one of the flattened sides has 100% of that surface region covered by a pigmented coating composition, the percentage of total surface area covered by the pigmented coating composition will be below 50%. Similarly, in another example, a hard candy substrate can comprise two confectionery masses such as a two-layer hard candy substrate with each confectionery mass having an exposed surface region. In this example, having 100% of one of the surface regions of one of the confectionery masses will provide a confectionery composition where the pigmented coating composition covers less than 100% of the total surface area.

In some embodiments, the hard candy substrates of the confectionery compositions can be produced by batch processes. Such hard candy substrates may be prepared using conventional apparatus such as fire cookers, cooking extruders, and/or vacuum cookers. In some embodiments, the bulk sweetener (sugar or sugar free) and a solvent (e.g., water), are combined in a mixing vessel to form a slurry. The slurry is heated to about 70° C. to 120° C. to dissolve any sweetener crystals or particles and to form an aqueous solution. Once dissolved, heat and vacuum are applied to cook the batch and boil off water until a residual moisture of less than about 4% is achieved. The batch changes from a crystalline to an amorphous, or glassy, phase. Optional additives, such as coloring agents, flavorants, and the like can then be admixed in the batch by mechanical mixing operations. The batch is then cooled to about 50° C. to 10° C. to attain a semi-solid or plastic-like consistency.

The optimum mixing required to uniformly mix flavors, colorants and other additives during manufacturing of hard candy substrates is determined by the time needed to obtain a uniform distribution of the materials. Normally, mixing times of from four to ten minutes have been found to be acceptable. Once the candy mass has been properly tempered, it may be cut into workable regions or formed into desired shapes such as drops, lozenges, lollipops, etc. having the desired weight and dimensions. A variety of forming techniques may be utilized depending upon the shape and size of the final product desired. Once the desired shapes are formed, cool air is applied to allow the comestibles to set uniformly, after which they are wrapped and packaged.

Alternatively, various continuous cooking processes utilizing thin film evaporators and injection ports for incorporation of ingredients are known in the art and can be used as well.

The apparatus useful in accordance with the present invention comprise cooking and mixing apparatus well known in the confectionery manufacturing arts, and selection of specific apparatus will be apparent to one skilled in the art.

In some embodiments, the pigmented coating composition is applied to a surface of the hard candy substrate after individual pieces of the hard candy substrate are formed while in other embodiments, the pigmented coating composition is applied to a surface of the hard candy substrate before individual pieces of the hard candy substrate are formed. For example, in some embodiments, a hard candy substrate is formed into a suitable shape for an individual lollipop piece and then the pigmented coating composition is applied to a surface of the individual lollipop piece. In other embodiments, a hard candy substrate is formed into a rope prior to forming individual pieces of the hard candy substrate and the pigmented coating composition is applied to a surface of the rope.

In some embodiments, a pattern is created when the pigmented coating composition is applied to the hard candy substrate. Such patterns can include, but are not limited to, a striped pattern, a swirled pattern, a random pattern, or combinations thereof. These patterns can be created by any method known in the art. Such methods can include but are not limited to spraying, brushing, rolling, dipping, stenciling, and combinations thereof. As used herein, the term “spraying” refers to application methods involving any type of nozzle known in the art. For example, spraying can include, but is not limited to atomization, drizzling, stripping, splattering, and the like. In one embodiment, a striped pattern can be created by spraying where the spraying operation includes dispensing the pigmented coating composition through a nozzle in a stream such that the stream forms a substantially linear stripe on a surface of the hard candy substrate. In another embodiment, a swirled pattern is created by spraying where the spraying operation includes dispensing the pigmented coating composition through a nozzle in a stream such that the stream forms a substantially non-linear swirl on a surface of the hard candy substrate. In yet another embodiment, a random pattern is created by spraying where the spraying operation includes intermittent dispensing of the pigmented coating composition onto a surface of the hard candy substrate to form spots.

In some embodiments, the pigmented coating composition is applied using pigment applicator apparatus including, but not limited to, sprayers, brushes, rollers, dippers, stencils, and combinations thereof. These pigment applicators can include any such apparatus known in the art. For example, sprayers can include apparatus employing various nozzle types and configurations including rotary sprayers as described in U.S. Pat. No. 6,340,488 which is incorporated herein by reference in its entirety for all purposes and gravity feed nozzle arrangements as described in U.S. Pat. No. 3,971,853 which is incorporated herein by reference in its entirety for all purposes.

In other embodiments, brushes and/or rollers can be used to apply the pigmented coating composition to the hard candy substrate. For example, brushes of varying bristle configurations to which the pigmented coating composition is fed or applied can be positioned so as to contact a surface of the hard candy substrate thereby applying the pigmented coating composition. In other examples, rollers including various embossed or debossed patterns to which the pigmented coating composition is fed or applied can be positioned so as to contact a surface of the hard candy substrate thereby applying the pigmented coating composition. Depending on the bristle and/or roller configuration and positioning of the brushes and/or rollers, various patterns can be created on a surface of the hard candy substrate. In some embodiments, sprayers are combined with brushes and/or rollers such that a sprayer applies the pigmented coating composition and a brush and/or roller manipulates the pigmented coating composition to form a pattern. In other embodiments involving a combination of sprayers and brushes and/or rollers, both sprayers and brushes and/or rollers can apply pigmented coating compositions. In some such embodiments, the pigmented coating compositions can be the same while in other embodiments, the pigmented coating compositions can be different.

In other embodiments, dippers can be used to apply the pigmented coating composition. For example, a dipper to which the pigmented coating composition is fed or applied can be positioned so that the hard candy substrate is at least partially submerged in the pigmented coating composition thereby applying the pigmented coating composition. In some embodiments, the dipper enrobes the hard candy substrate in the pigmented coating composition. In some such embodiments, apparatus such as brushes can be used to manipulate the surface area coverage of the hard candy substrate so that the substantially less than 75% of the total surface area of the hard candy substrate is covered with the pigmented coating composition.

In still other embodiments, stencils can be used to apply to pigmented coating composition. For example, a stencil formed from a solid material into which a pattern has been cut and to which the pigmented coating composition is fed or applied can be positioned on or near a surface of the hard candy substrate thereby applying the pigmented coating composition. In other examples, a stencil to which the pigmented coating composition is not fed or applied can be positioned on or near the surface of the hard candy substrate and a pigment applicator such as a sprayer or roller can apply the pigmented coating composition to form the pattern provided by the stencil. In some embodiments, the solid portion of the stencil blocks or limits the amount of pigmented coating composition which is applied to the hard candy substrate. For example, the stencil can block at least 25% of the total surface area of the hard candy substrate with the result that substantially less than 75% of the total surface area is covered with pigmented coating composition.

### EXAMPLES

The features and advantages of the present invention are more fully shown by the following examples which are provided for purposes of illustration, and are not to be construed as limiting the invention in any way.

### Examples A-B

TABLE 1
Hard Candy Substrates

| | Example A | Example B |
|---|---|---|
| | % w/w by weight of | % w/w by weight of |
| Ingredient | hard candy substrate | hard candy substrate |
| Sucrose | 40-60 | |
| 42 DE Corn Syrup | 40-60 | |
| Isomalt | | 40-60 |
| Maltitol Syrup | | 40-60 |
| Acid | 0.5-4.0 | 0.5-4.0 |
| Color | 0.005-1.5 | 0.005-1.5 |
| Flavor | 0.03-0.8 | 0.03-0.8 |

To form the confectionery product, the sucrose and glucose syrup or isomalt and maltitol syrup are dissolved in water, mixed, and heated to about 145° C. The cooked candy masses are then placed on cooling tables where color, acid, and flavor are mixed with kneading. Individual pieces of the finished hard candy substrate products are formed by any known process including molding, forming, etc. To create hard candy substrate surfaces with more porosity, the hard candy substrate can be mixed with additional kneading and/or can be pulled. To create hard candy substrate surfaces with less porosity, the hard candy substrate can be deposited into molds.

### Examples C-F

TABLE 2
Pigmented Coating Compositions

| Ingredient | C | D | E | F |
|---|---|---|---|---|
| Color | | 2-10 | | |
| Pearlescent | 15-25 | 15-25 | 15-25 | 15-25 |
| Pigment | | | | |
| Fluid Carrier- | q.s. | | | q.s. |
| vegetable oil | | | | |
| Powder Carrier- | | q.s. | q.s. | |
| mannitol | | | | |
| High-Intensity | | | 0.001-0.01 | 0.001-0.01 |
| Sweetener | | | | |
| Flavor | 0.001-0.01 | | | 0.001-0.01 |
| Sensate | | 0.001-0.01 | | 0.001-0.01 |

All percentages shown above are w/w percentages by weight of the pigmented coating composition.

To form the pigmented coating composition, the ingredients shown in Table 2 are mixed together in any suitable mixing equipment. Mixing is allowed to proceed until a homogeneous blend is obtained.

The pigmented coating compositions in Examples C-F can be applied to the individual pieces of the formed hard candy substrates in Examples A-B by any suitable method including spraying, brushing, rolling, dipping, stenciling, and combinations thereof as described above. The amount of total surface area covered by such application can be less than substantially 75%, 70%, 65%, 60%, 55%, 50%, 45%, 40%, 35%, 30%, 25%, 20%, 15%, 10%, 5%, or 2%.

Alternatively, the pigmented coating compositions in Examples C-F can be applied to the hard candy substrates in Examples A-B prior to forming by any suitable method including spraying, brushing, rolling, dipping, stenciling, and combinations thereof as described above. The amount of total surface area covered by such application can be less than substantially 75%, 70%, 65%, 60%, 55%, 50%, 45%, 40%, 35%, 30%, 25%, 20%, 15%, 10%, 5%, or 2%.

1. A confectionery composition comprising:
a hard candy substrate having a total surface area, and
a pigmented coating composition including a pearlescent pigment and a fluid carrier,
wherein said pigmented coating composition covers less than substantially 75% of said total surface area.

2. The confectionery composition as in claim 1 wherein said pigmented coating composition covers less than substantially 50% of said total surface area.

3. The confectionery composition as in claim 1, wherein said hard candy substrate is a lollipop.

4. The confectionery composition as in claim 1, wherein said total surface area includes more than one surface region and wherein said pigmented coating composition covers 100% of one of said more than one surface region.

5. The confectionery composition as in claim 1, wherein said pigmented coating composition includes at least one food color selected from the group consisting of artificial colors, natural colors, and combinations thereof.

6. The confectionery composition as in claim 1, wherein said pigmented coating composition includes at least one tastant selected from the group consisting of flavors, sweeteners, acids, salts, potentiators, and combinations thereof.

7. The confectionery composition as in claim 1, wherein said pigmented coating composition includes at least one active selected from the group consisting of medicaments, nutraceuticals, pharmaceuticals, micronutrients, and combinations thereof.

8. The confectionery composition as in claim 1, wherein said fluid carrier is selected from the group consisting of oil, fat, wax, shellac, and combinations thereof.

9. The confectionery composition as in claim 1, wherein said pigmented coating composition is capable of creating a pattern selected from the group consisting of a striped pattern, a swirled pattern or a random pattern, or any combination thereof.

10. A method of applying pigment to a confection comprising the steps of
providing a hard candy substrate having a total surface area,
providing a pigmented coating composition including a pearlescent pigment and a fluid carrier, and
applying said pigmented coating composition to said hard candy substrate such that said pigmented coating composition covers less than substantially 75% of said total surface area, and wherein said applying step involves a process other than panning.

11. (canceled)

12. The method as in claim 10, wherein said pigmented coating composition covers less than substantially 50% of said total surface area.

13. The method as in claim 10, wherein said applying includes spraying and wherein said spraying includes intermittent spraying.

14. The method as in claim 10, wherein said applying includes brushing and wherein said brushing includes providing one or more brushes including a set of bristles wherein said bristles are selected from the group consisting of bristles arranged to include spaces, bristles arranged to include spaces that are at least 1 mm apart, and combinations thereof.

15. The method as in claim 10, wherein said applying includes stenciling and said stenciling includes providing one or more stencils wherein said one or more stencils block at least 25% of said total surface area during said applying.

16. The method as in claim 10, wherein said applying creates a pattern selected from the group consisting of a striped pattern, a swirled pattern or a random pattern, or any combination thereof.

17. The method as in claim 10, wherein said applying step is selected from the group consisting of spraying, brushing, rolling, dipping, stenciling, and combinations thereof.

18. The method as in claim 10, including a forming step prior to said applying step.

19. The method as in claim 10, including a forming step subsequent to said applying step.

20. An apparatus for applying pigment to a confection comprising a confectionery substrate conveyor and a pigment applicator, wherein said pigment applicator is selected from the group consisting of sprayers, brushes, rollers, dippers, stencils, and combinations thereof and wherein said pigment applicator covers a confectionery substrate having a total surface area with less than substantially 75% coverage of said total surface area.

21. The apparatus as in claim 20, wherein said pigmented coating composition covers less than substantially 50% of said total surface area.

22. The apparatus as in claim 20, wherein said applying includes spraying and wherein said spraying includes intermittent spraying.

23. The apparatus as in claim 20, wherein said applying includes brushing and wherein said brushing includes providing one or more brushes including a set of bristles wherein said bristles are selected from the group consisting of bristles arranged to include spaces, bristles arranged to include spaces that are at least 1 mm apart, and combinations thereof.

24. The apparatus as in claim 20, wherein said applying includes stenciling and said stenciling includes providing one or more stencils wherein said one or more stencils block at least 25% of said total surface area during said applying.

25. The apparatus as in claim 20, wherein said applying creates a pattern selected from the group consisting of a striped pattern, a swirled pattern or a random pattern, or any combination thereof.
